(19) United States
(12) Patent Application Publication
(10) Pub. No.: US 2006/0234914 A1
(43) Pub. Date: Oct. 19, 2006

(54) Fragments of proinsulin c-peptide

(75) Inventors: John Wahren, Stockholm (SE); Hans Jornvall, Stockholm (JP)

Correspondence Address: MORRISON & FOERSTER LLP, 425 MARKET STREET, SAN FRANCISCO, CA, 94105-2482, US

(73) Assignee: Creative Peptides Sweden AB, Stockholm (SE)

(21) Appl. No.: 10/524,347

(22) PCT Filed: Aug. 14, 2003
(86) PCT No.: PCT/GB03/03571

§ 371 Date: Apr. 5, 2006

(30) Foreign Application Priority Data
Aug. 14, 2002 (GB) 0218970.2

(51) Int. Cl. A61K 38/28; A61K 38/10; C07K 7/08

(52) U.S. Cl. 514/3; 530/303; 530/327; 514/14

(57) ABSTRACT

The present invention relates to peptides being the N-terminal fragment of human proinsulin C-peptide and having the sequence EAEDLQVGQVEL (SEQ ID No: 2) or a fragment or peptide derivative thereof retaining the functional ability of the N-terminal fragment to contribute to C-peptide activity, wherein said fragment or peptide derivative comprises two acidic amino acid residues and is capable of adopting a conformation where said two acidic amino acid residues are spatially separated from one another by a distance of 9-14 Å between the α-carbons thereof; and wherein said peptide derivative does not include native C-peptide of any species nor human C-peptide 1-15, 1-24 or 1-26 or rat C-peptide 1-26. The invention also relates to peptides having an amino acid sequence comprising (i) the N-terminal fragment of human insulin C-peptide having the sequence EAEDLQVGQVEL (SEQ ID NO. 2) or (ii) a fragment or peptide derivative of amino acid sequence SEQ ID NO. 2 retaining the functional ability of said N-terminal fragment to contribute to C-peptide activity, wherein said fragment or peptide derivative comprises two acidic amino acid residues and is capable of adopting a conformation wherein said two acidic amino acid residues are spatially separated from one another by a distance of 9-14 Å between the a-α-carbons thereof; said peptide having C-peptide activity, but not including native C-peptide of any species nor human C-peptide 1-15, 1-24 or des 13-17. The invention further relates to peptides having the formula Xn-Y-Xm-Y-Xp where X is any amino acid, Y is an acidic amino acid, n=0-6, m=5-9 and p=0-6. First and second medical indications, pharmaceutical compositions and products for use as a combined preparation are also covered by the present invention.

The present invention relates to fragments of the proinsulin C-peptide, particularly N-terminal fragments, and their use in the treatment of diabetes and diabetic complications.

Insulin-dependent diabetes mellitus (IDDM), generally synonymous with type 1 diabetes, is the classical, life-threatening form of diabetes, the treatment of which was revolutionized by the discovery of insulin in 1922. The prevalence of IDDM is unfortunately widespread throughout much of the world and hence IDDM represents a serious condition with a significant drain on health resources.

The etiology of IDDM is multifactorial and not yet entirely clear. However it is characterised by a partial or complete destruction of the pancreatic beta cells. In the acute phase of IDDM insulin deficiency is thus the dominating pathophysiological feature.

After starting insulin treatment many patients enjoy good blood glucose control with only small doses of insulin. There is an early phase, the “honeymoon period”, which may last a few months to a year and which probably reflects a partial recovery of beta cell function. This is, however, a temporary stage and ultimately, the progressive destruction of the beta cells leads to complete cessation of insulin secretion and increasing requirements for exogenous insulin.

While the short term effects of hypoinsulinemia in the acute phase of IDDM can be well controlled by insulin administration, the long term natural history of IDDM is darkened by the appearance in many patients of potentially serious complications known as late, or late onset complications. These include the specifically diabetic problems of nephropathy, retinopathy and neuropathy. These conditions are often referred to as microvascular complications even though microvascular alterations are not the only cause. Atherosclerotic disease of the large arteries, particularly the coronary arteries and the arteries of the lower extremities, may also occur.

Nephropathy develops in approximately 35% of IDDM patients, particularly in male patients and in those with onset of the disease before the age of 15 years. Diabetic nephropathy is characterized by persistent albuminuria secondary to glomerular capillary damage, a progressive reduction of the glomerular filtration rate and eventually, end stage renal failure.

The prevalence of diabetic retinopathy is highest among young-onset IBDM patients and it increases with the duration of the disease. Proliferative retinopathy is generally present in about 25% of the patients after 15 years duration and in over 50% after 20 years. The earliest lesion of diabetic retinopathy is a thickening of the capillary basement membrane, followed by capillary dilatation and leakage and formation of microaneurysms. Subsequently, occlusion of retinal vessels occurs resulting in hypoperfusion of parts of the retina, oedema, bleeding and formation of new vessels as well as progressive loss of vision.

Diabetic neuropathy includes a wide variety of disturbances of somatic and autonomic nervous function. Sensory neuropathy may cause progressive loss of sensation or, alternatively, result in unpleasant sensations, often pain, in the legs or feet. Motor neuropathy is usually accompanied by muscle wasting and weakness. Nerve biopsies generally show axonal degeneration, demyelination and abnormalities of the vasa nervorum. Neurophysiological studies indicate reduced motor and sensory nerve conduction velocities. Autonomic neuropathy afflicts some 40% of the patients with IDDM of more than 15 years duration. It may evolve through defects in thermoregulation, impotence and bladder dysfunction followed by cardiovascular reflex abnormalities. Late manifestations may include generalized sweating disorders, postural hypotension, gastrointestinal problems and reduced awareness of hypoglycemia. The latter symptom has grave clinical implications.

A number of theories have been advanced with regard to possible mechanism(s) involved in the pathogenesis of the different diabetic complications but this has not yet been fully elucidated. Metabolic factors may be of importance and it has been shown that good metabolic control is accompanied by significantly reduced incidence of complications of all types. Nevertheless, after 7-10 years of good metabolic control, as many as 15-25% of the patients show signs of beginning nephropathy, 10-25% have symptoms of retinopathy and 15-20% show delayed nerve conduction velocity indicating neuropathy. With longer duration of the disease the incidence of complications increases further. There is thus a significant clinical need for the control and management of these diabetic complications.

Proinsulin C-peptide is a part of the proinsulin molecule which, in turn, is a precursor to insulin formed in the beta cells of the pancreas. For a long time it was believed that C-peptide (known variously as C-peptide or proinsulin C-peptide) had no role other than as a structural component of proinsulin, facilitating correct folding of the insulin part. However, it has in more recent years been recognised that C-peptide has a physiological role as a hormone in its own right (Wahren et al., (2000), Am. J. Physiol. Endocrinol. Metab, 278, E759-E768). In diabetic patients, it alleviates renal dysfunction, improves blood flow in several tissues, ameliorates nerve functional impairments and is believed to delay or prevent the onset of late complications (Wahren et al., (2000) supra; Wahren and Johansson (1998), Horm. Metab. Res. 30, A2-A5). Indeed, C-peptide has been proposed for use in the treatment of diabetes in EP 132769 and in SE460334 for use in combination with insulin in the treatment of diabetes and prevention of diabetic complications.

A receptor for C-peptide has not yet been defined but molecular studies using fluorescence correlation spectroscopy show specific binding of human C-peptide to cell membranes from a number of tissues (Rigler et al., (1999) PNAS USA 96, 13318-13323; Pramanik et al., (2001) BBRC 284, 94-98) and intracellular calcium measurements show that C-peptide increases the intracellular level of calcium (Ohtomo et al., (1996) Diabetologia 39, 199-205; Kunt et al., (1998) Diabetes 47, A30; Shafqat et al., (2002) Cell Mol. Life Sci. 59, 1185-1189), thus supporting a hormone function for C-peptide.

Further work has shown that the C-terminal pentapeptide fragment of C-peptide has similar physiological and molecular effects to C-peptide itself, suggesting that this segment is an essential part of C-peptide (Wahren et al., 2000, supra; Rigler et al., 1999, supra; Ohtomo et al., 1998, Diabetologia 41, 287-291; Pramanik et al., 2001, supra; Shafqat et al, 2002, supra). WO 98/13384 proposes the use of this C-terminal pentapeptide, and other C-terminally located peptide fragments of C-peptide in the treatment of diabetes and diabetic complications.

However, the mechanism of action of C-peptide and the identity of its important active sites is not entirely clear cut. Thus, various studies suggest that more than one signalling pathway, and perhaps even more than one receptor, may be involved.

A number of actions of C-peptide appear to be mediated via G-protein-coupled pathways, as indicated by pertussis toxin inhibition of those actions. Thus, pertussis toxin inhibits C-peptide stimulation of Na⁺K⁺ATPase activity, calcium influx and activation of MAP kinases. Interestingly, it also interferes with C-peptide binding to cell membranes (Rigler et al., supra). Moreover, activation of protein kinase C and phosphoinositide 3-kinase P13-K) seems to be involved in C-peptide induced phosphorylation of MAP kinases (Kitamura et al., (2001), Biochem. J. 355, 123-129).

Interactions between C-peptide and receptors with catalytic activity are indicated by results showing that C-peptide attenuates protein tyrosine phosphatase activity (Li et al., (2001), B.B.R.C. 280, 615-619). Protein tyrosine phosphatases inactivate the insulin signalling pathway by dephosphorylation of the insulin receptor, insulin receptor substrates and MAP kinases. Hence, C-peptide and insulin might have a synergistic effect on the insulin signalling pathway at the level of the insulin receptor. This is further corroborated by the recent finding that C-peptide at physiological concentrations mimics insulin effects in myoblasts; it activates insulin receptor tyrosine kinase, insulin receptor substrate-1 tyrosine phosphorylation, PI3-K activity, and MAP kinase phosphorylation (Grunberger et al., (2001), Diabetologia, 44, 1247-1257). If C-peptide is added in the presence of high insulin concentrations, no further effects are observed, indicating that C-peptide and insulin may use the same signalling pathway. These authors suggested that low C-peptide levels enhance insulin effects, while at supra-physiological concentrations C-peptide blunts insulin effects. However, C-peptide, unlike insulin, does not activate Akt (protein kinase B), suggesting that C-peptide also works via mechanisms distinct from those of insulin. C-peptide-induced stimulation of glycogen synthesis in the myoblasts was blocked by Wortmannin, an inhibitor of PI3-K activity, but not by pertussis toxin (Grunberger, (2001), supra). In contrast to these findings, Zierath et al., 1996, Diabetologia 39, 421-432, found that C-peptide stimulates glucose transport in human muscle strips, and that these effects were not mediated via the insulin receptor or tyrosine kinase activation.

It has also been speculated that C-peptide may interact with ligand-gated ion channels coupled to glutamate receptors, based, for example, on the observation that C-peptide, in common with other Glu-terminated peptides, may antagonise the N-Methyl-D-Aspartate (NDMA) receptor (Bourguignon et al., (1994), Endocrinology 134, 1589-1592). Further, free Glu has been shown to have some C-peptide activity in several assays (Johannsson et al., (2002) Biochem. Biophy. Res. Commun 295, 1035-1040). However, this has yet to be confirmed.

It has also been suggested that the effects of C-peptide may be mediated by direct membranotropic mechanisms instead of by classical receptor-ligand interactions (Ido et al., (1997), Science 277, 563-566). However, such observations are not entirely consistent with other studies showing the absence of the hallmarks of traditional pore-forming peptides in C-peptide, (Steiner et al., (1997), Science 277-, 531-532; Henriksson et al., (2000), Cell Mol. Life Sci. 57, 337-342) and again this is yet to be fully resolved.

Thus, the above studies indicate that there may be a diversity of C-peptide action, with perhaps different effects being mediated in different ways. Thus, for example, receptor-mediated physiological effects may take place at or below the physiological C-peptide concentration (0.5-1.5×10⁻⁹M), whereas supra-physiological concentrations might induce non-specific effects. Where receptor action is concerned, as mentioned above, the experimental data tends to suggest that other receptors and/or signalling pathways may be involved in addition to a G-protein coupled receptor(s). We further suggest that such diverse actions may be mediated in different ways by different portions, or segments, of the C-peptide molecule.

Thus, although clinical utility has been demonstrated for C-peptide and its C-terminal fragment, the above studies open up the possibility that, through further understanding of how C-peptide works, improvements may be achieved in C-peptide-based therapy of diabetes and its complications, and the present invention is directed to this aim.

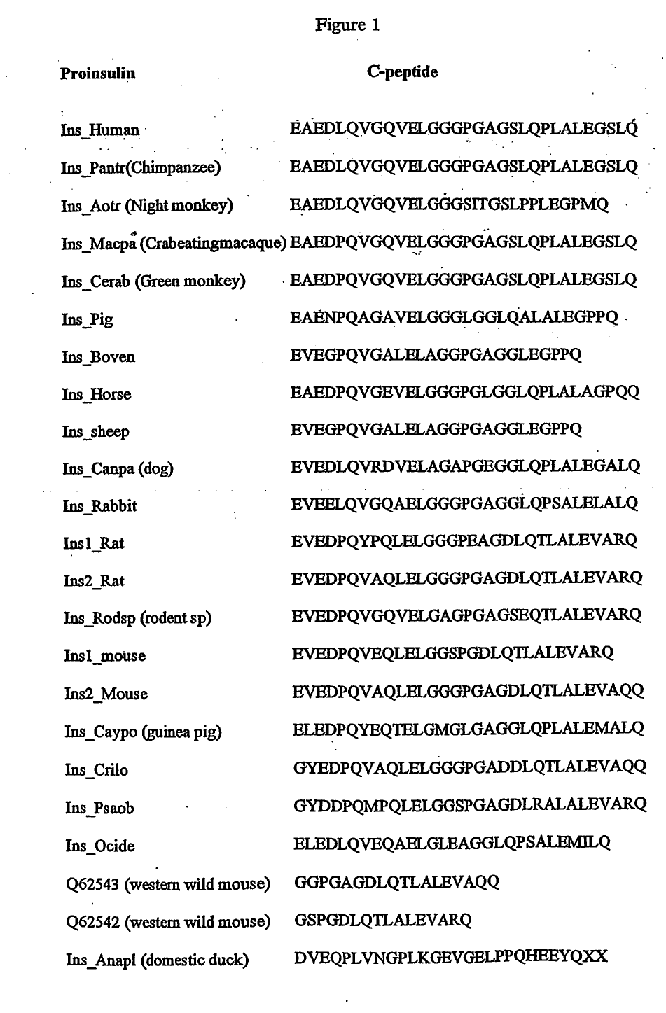

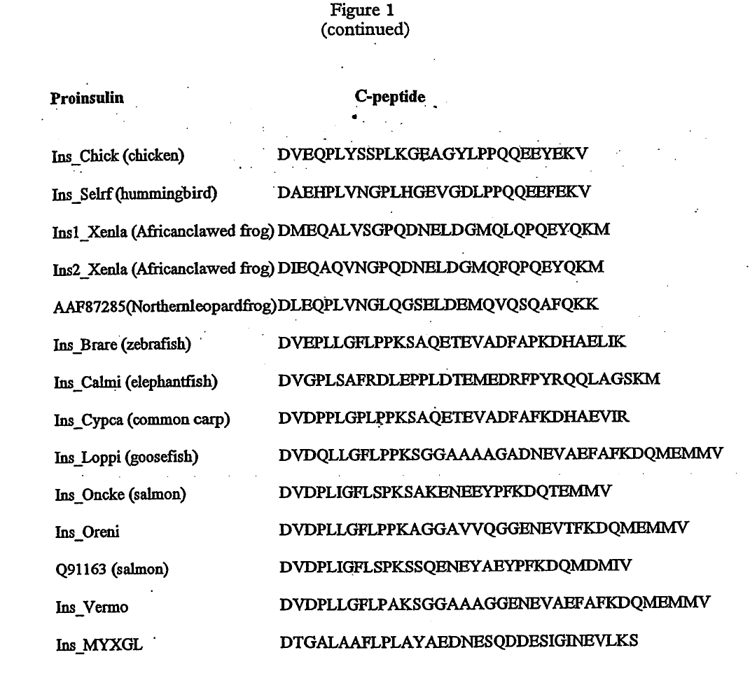

Proinsulin, or large parts of it, are known in 37 different variants, representing 33 different species, ranging from Atlantic hagfish, Myxine glutinova, to human. Whilst the insulin segments (i.e. the A and B chains of proinsulin) are well conserved between species, C-peptide is much more highly variable, showing not only sequence variation, but also several internal deletions, making the length of C-peptide variable (see FIG. 1).

Human C-peptide is a 31 amino acid peptide having the following sequence: EAEDLQVGQVELGGGPGAGSLQPLALEGSLQ (SEQ ID. NO. 1).

C-peptide has thus hitherto been regarded as a poorly conserved peptide. It will be seen, however, that when different groups of C-peptide are compared, mammalian C-peptides for example, higher levels of conservation can be seen and conserved residues can be identified. It will further be seen that C-peptide can be ascribed a tripartite overall structure, with more conserved N- and C-terminal segments and a more variable mid-sequence, or internal, portion. Thus, in the case of human C-peptide the N-terminal segment can be regarded as residues 1-12, the mid-portion as residues 13-26, and the C-terminal segment as residues 27-31.

In water, C-peptide is devoid of detectable stable secondary structure, and thus appears to be unordered. However, under artificial conditions in trifluoroethanol (TFE) the N-terminal 11 residues can be induced to form an α-helical structure (Henriksson et al., (2000), Cell.

Mol. Life Sci. 57, 337-342). However, no importance, functional or otherwise, has been ascribed to this.

As mentioned above, the C-terminal segment has been recognised in the art to have both binding and physiological activity, and clinical utility (see e.g. WO 98/13384 and Ohtomo et al., (1998), supra). The mid-portion also has been shown to have molecular and physiological effects (see e.g. Ido et al. and Ohtomo et al., (1998), supra) and has also. been proposed in WO 98/13384 to have clinical utility. Ido et al., have speculated that the mid-portion may mediate its effects through membrane interactions, as mentioned above, although this is still to be confirmed and rather contradicted by other studies (Henriksson et al, supra). In any event, the available data, for example showing that the mid-portion peptide fragment comprising amino acid residues 11-19 of human C-peptide does not displace cell membrane-bound human C-peptide (Pramanik et al., (2001), supra), suggests that the mechanisms of the mid-portion segment are different from those of the C-terminal segment. Up till now, however, the N-terminal segment (or portion) of C-peptide has not been thought to be important for activity and indeed N-terminal fragments or portions containing the N-terminal segment have not been found to show any reproducible activity (see e.g. Ohtomo et al., (1998), supra).

We have now surprisingly found, however, that although not active on its own, the N-terminal segment is functionally important, and makes an important contribution to the activity of C-peptide. This has been elucidated through a detailed study of the structural basis for C-peptide activity, and has been confirmed by experimental data showing the importance of certain residues in the N-terminal segment for C-peptide activity. It is now proposed, therefore, that the N-terminal fragment has a utility in C-peptide-based therapies, for example either in conjunction with an active C-terminal fragment, or as part of a modified C-peptide molecule.

Furthermore, it has also been shown that substitution of certain amino acid residues in the N-terminal region may increase or decrease C-peptide activity. This, thus, opens up the possibility of modifying the N-terminal sequence of C-peptide in order to modulate its activity, namely the construction of peptide derivatives of the N-terminal region of C-peptide, or peptide derivatives of C-peptide (e.g. comprising modifications in the N-terminal region) which may have e.g. altered C-peptide activity or which may otherwise be useful in C-peptide based therapies.

In particular, we have now found that Glu residues 3 and 11 of human C-peptide located in the N-terminal region are particularly important for activity, in conjunction with Glu 27 of the C-terminal part, which has previously been reported to be important for activity of the C-terminal fragment and C-peptide as a whole (Pramanik et al., (2001), supra).

Whilst not wishing to be bound by theory, it is believed that these three acidic amino acid residues are important for receptor binding, and/or for C-peptide binding activities and that the reason why the C-terminal fragment is active on its own is that it can adopt a structure similar to that of the N-terminal fragment at one or both of the conserved N-terminal region Glu residues (Glu3 and/or Glu11), and thus may bind to the receptor/binding proteins not only at its own site for the C-terminal segment, but also at a site for the N-terminal segment. In this way, the C-terminal fragment can mimic the receptor interactions of the intact C-peptide.

We further find evidence that on interacting with its receptor, the N-terminal segment is induced to form an α-helical structure, and hence we now propose for the first time that such a structure is functionally important. We have found in this regard that the critical Glu residues Glu3 and Glu11, together with other conserved amino acids in the N-terminal segment (e.g. Gln 6 and Val 7) are located on one side of an α-helix. We thus propose a model for receptor or protein interaction, wherein the N-terminal segment of C-peptide is induced into an α-helical conformation, having a conserved surface encompassing one side of the helix and presenting two acidic (Glu) residues for receptor or protein binding.

In one aspect, the present invention thus provides a peptide, being the N-terminal fragment of human proinsulin C-peptide, and having the sequence

| E A E D L Q V G Q V E L | (SEQ ID. NO. 2) |
|---|---|
| 1 2 3 4 5 6 7 8 9 10 11 12 | |

or a fragment or peptide derivative thereof retaining the functional ability of said N-terminal fragment to contribute to C-peptide activity, wherein said fragment or peptide derivative comprises two acidic amino acid residues and is capable of adopting a conformation where said two acidic amino acid residues are spatially separated from one another by a distance of 9-14 Å between the α-carbons thereof, preferably 10-13 Å (e.g. 10-12.6 Å); and wherein said peptide derivative is no more than 14 amino acids in length or wherein said derivative does not include native C-peptide of any species (as shown in FIG. 1) nor human C-peptide 1-15, 1-24 or 1-26 or rat C-peptide 1-26.

Alternatively, the spatial separation between the two acidic residues can be considered in terms of the number of intervening amino acids in the primary sequence or structure. Thus, the spacing between the two acidic amino acid residues can be 5-9 amino acid residues, preferably 5-8 or 6-8 amino acid residues and most preferably 7 amino acids as in SEQ. ID. NO. 2.

More particularly, the peptide fragment or derivative is capable of adopting an α-helical conformation, and even more particularly the two said acidic amino acid residues are located on one side of said α-helix. This requirement for capability of forming an α-helix means that the peptide fragment or derivative may form an α-helix under appropriate conditions, for example in a suitable (e.g. structure-promoting) solvent such as TFE (see Henriksson et al., supra), or when interacting with a receptor or binding protein. This requirement may thus readily be tested for under appropriate test conditions using standard analytical techniques, for example NMR and circular dichroism in TFE as described by Henriksson et al.

In a more particular embodiment, the peptide derivative comprises also further amino acid residues of the N-terminal fragment or segment of human C-peptide, which are located on said one side of the said α-helix such that said helix presents a “conserved surface” encompassing one side of the helix. In particular, said “conserved surface” comprises residues, in addition to the two acidic amino acid residues (corresponding to Glu 3 and Glu 11 of human C-peptide), Gln 6 and/or Val 7 of human C-peptide or equivalent or corresponding residues.

Preferably, the minimum length of the peptide of the invention is 7 amino acids and the peptide is capable of forming an α-helix. More preferably, the minimum length is 8, 9, 10, 11 or 12 amino acids.

By “equivalent” or “corresponding” residues is meant the residues which occur in positions equivalent or corresponding to the stated positions, e.g. as identified above and below, in the human C-peptide, i.e. the equivalent or corresponding residues in other native forms of C-peptide e.g. in other species (see e.g. FIG. 1).

Even more particularly, the two said acidic amino acid residues of said peptide derivative are capable of interacting with a third acidic residue (namely Glu 27) when supplied as an additional peptide (e.g. the C-terminal pentapeptide of human C-peptide), or indeed in the context of a modified peptide comprising said peptide derivative sequence and said additional peptide sequence, in stimulating C-peptide activity. Thus, in the context of such a modified peptide, or when said peptide derivative is used in conjunction with said additional peptide, the spatial separation between said two acidic residues and said third acidic residue (e.g. Glu 27) is in the ranges specified above, i.e. the spatial separation is preferably equidistant between the three acidic residues (distance between each of the three acidic residues one with the others is the same).

The term “peptide derivative” as used herein means that the derivative in question has a peptide structure. In other words the derivative is a peptide derived from or corresponding to or a variant of the N-terminal fragment of human C-peptide having sequence modifications as compared to the native human N-terminal C-peptide fragment sequence. Such modifications may be one or more amino acid deletions, additions, insertions and/or substitutions. Chemical modification of the peptide structure is not precluded e.g. by glycosylation as long as the structure of the derivative remains essentially peptide in nature. As mentioned above, modification of an amino acid sequence may be by amino acid substitution, for example an amino acid may be replaced by another which preserves the physicochemical character of the peptide (e.g. A may be replaced by G or vice versa, V by A, L or G; E by D or vice versa; and Q by N). Generally, the substituting amino acid has similar properties e.g. hydrophobicity, hydrophilicity, electronegativity, bulky side chains etc. to the amino acid being replaced.

Additional variants may include amino and/or carboxyl terminal fusions as well as intrasequence insertions of single or multiple amino acids. Longer peptides may comprise multiple copies of one or more of the peptide sequences.

Insertional amino acid sequence variants are those in which one or more amino acid residues are introduced at a site in the protein. Deletional variants are characterised by the removal of one or more amino acids from the sequence.

The term “functional ability” as used herein means that the fragment or derivative is capable of functioning as the N-terminal fragment of human proinsulin C-peptide, i.e. can substitute for the N-terminal fragment itself, for example in the context of the intact human C-peptide itself, or when used in combination with the active C-terminal pentapeptide fragment EGSLQ (SEQ ID-NO. 3). Thus the fragment or derivative retains the functional characteristics of the N-terminal fragment, and particularly the functional property of the N-terminal fragment in contributing to C-peptide activity.

By “contributing” is meant that in the context of an active C-peptide molecule (e.g. intact human C-peptide or an active sequence-modified derivative thereof), the substitution of the fragment/derivative of the invention for the native N-terminal segment does not abrogate (or substantially abrogate) C-peptide activity. Thus, detectable, and preferably clinically or physiologically significant levels of C-peptide activity are retained. In other words activity is retained at levels similar or close to levels of native C-peptide, or any reduction in activity is such as for activity still to be detectable, or useful, e.g. the peptide retains at least 25% (and more preferably at least 30, 40, 50, 60, 70, 75, 80 or 90% of the activity of the native, (e.g. “parent”) molecule i.e. prior to the substitution. Similarly, and analogously, such contribution might be assessed in the context of determining the activity of the N-terminal fragment or derivative admixed with an “active” C-peptide fragment e.g. an active C-terminal fragment e.g. a C-terminal pentapeptide such as the human C-terminal pentapeptide.

The term “C-peptide activity” as used herein means any activity, exhibited by a native C-peptide, whether a physiological response exhibited in an in vivo or in vitro test system, or any biological activity or reaction mediated by a native C-peptide, for example in an enzyme assay or in binding to test tissues or membranes.

Thus, it is known that C-peptide increases the intracellular concentration of calcium. An assay for C-peptide activity can thus be by assaying for changes in intracellular calcium concentrations upon addition or administration of the peptide (e.g. fragment or derivative) in question. Such an assay is described in for example in Ohtomo et al., (1996), supra, Kunt et al., supra; Shafqat et al., supra and in Example 1 below.

Further, C-peptide has been found to induce phosphorylation of the MAP-kinases ERK 1 and 2 of a mouse embryonic fibroblast cell line (Swiss 3T3), and measurement of such phosphorylation and MAPK activation may be used to assess, or assay for C-peptide activity, as described for example by Kitamura et al., supra and in Example 2.

C-peptide also has a well known effect in stimulating Na⁺K⁺ATPase activity and this also may form the basis of an assay for C-peptide activity, for example as described in WO 98/13384 or in Ohtomo et al., (1996), supra or Ohtomo et al., (1998), supra.

An assay for C-peptide activity based on endothelial nitric oxide synthase (eNOS) activity is also described in Kunt et al., supra, using bovine aortic cells and a reporter cell assay.

Binding to particular cells may also be used to assess or assay for C-peptide activity, for example to cell membranes from human renal tubular cells, skin fibroblasts and saphenous vein endothelial cells using fluorescence correlation spectroscopy, as described for example in Rigler et al., supra, Henriksson et al., supra and Pramanik et al., supra. Finally, affinity tests based on measurements of protein binding may be used as activity tests of C-peptide.

The term “spatially separated” means the relative 3-D positions of the residues in question, i.e. the separation, or relative positions of the residues in a 3-D structure or conformation.

It will be clear from the above that the “fragments” of the N-terminal C-peptide fragment, according to this aspect of the present invention, as defined above, must comprise at least Glu 3 and Glu 11. Such “fragments” thus include

| | |
|---|---|
| A E D L Q V G Q V E L | (SEQ ID NO. 4) |
| E D L Q V G Q V E L | (SEQ ID NO. 5) |
| E D L Q V G Q V E | (SEQ ID NO. 6) |
| A E D L Q V G Q V E | (SEQ ID NO. 7) |
| E A E D L Q V G Q V E. | (SEQ ID NO. 8) |

However, by analogy with the active C-terminal fragments discussed above, it is also believed that short fragments of the N-terminal C-peptide fragments, which contain only 1 Glu residue may be active in their own right i.e. may exhibit C-peptide activity (i.e. similarly to that shown for the C-terminal pentapeptide of C-peptide). Thus, such short Glu-containing peptide fragments may be able to bind to the receptor(s) at the sites for both the N-terminal and C-terminal segments and thus mimic the receptor interactions of the intact C-peptide, as proposed for the C-terminal pentapeptide and other C-terminal fragments above.

A further separate, but related, aspect of the invention thus provides a peptide, being a fragment of the N-terminal segment of a proinsulin C-peptide (namely the N-terminal 1-12 amino acid residues), said fragment having no more than 6 amino acids and having C-peptide activity. This aspect also provides such peptides for use in therapy.

The proinsulin C-peptide may be any of the C-peptides shown in FIG. 1, but preferably will be a mammalian C-peptide. Accordingly, such short active peptides according to this aspect of the invention preferably include one of the Glu residues (i.e. one of Glu 3 and Glu 11) or corresponding or equivalent residue in other species (preferably an acidic residue e.g. Asp).

Representative short active fragments of this aspect of the invention thus include fragments (i.e. C-peptide residues) 1-6, 1-5, 1-4, 1-3, 2-7, 2-6, 2-5, 2-4, 4-7, 3-8, 3-7, 3-6, 3-5, 6-11, 7-12, 7-11, 8-12, 8-11, 9-12, 9-11, 10-12, particularly such fragments of human C-peptide. Preferred such short active fragments thus include, inter alia, human 3-7, E D L Q V (SEQ ID NO. 9), human 1-5, E A E D L (SEQ ID NO. 10), human 7-11, V G Q V E (SEQ ID NO. 11).

As mentioned above, it is considered that such short fragments have activity by virtue of including an acidic residue important for receptor interaction and being short enough to fit into the receptor binding pockets for both the N- and C-terminal segments of C-peptide. Thus, this then leads to a related, but broader aspect of the present invention, in which it is proposed that any short peptide (i.e. up to 6 residues long) containing at least one acidic residue may be able to mimic C-peptide activity, or more particularly to mimic C-peptide receptor interactions, and thus exhibit C-peptide activity.

In this aspect the present invention thus provides a peptide for use in therapy, said peptide being no more than 6 amino acid residues in length and comprising at least one acidic amino acid residue; with the proviso that said peptide is not ESGLQ (SEQ ID NO. 3), ELGGGP (SEQ ID NO. 12), ELGG (SEQ ID NO. 13), ELGGG (SEQ ID NO. 14) or EVARQ (SEQ ID NO. 15).

The therapy is preferably C-peptide therapy, as discussed further below. The peptide may be 2, 3, 4, 5 or 6 residues in length, preferably up to 5 residues (e.g. 2 to 5 or 3 to 5). The acidic residue may be any acidic residue as discussed further below, but preferably will be Glu or a derivative thereof, as discussed further below. Representative peptides of this aspect of the invention thus include e.g.

| | |
|---|---|
| EEEEEE; | (SEQ ID NO. 16) |
| EEEEE; | (SEQ ID NO. 17) |
| EEEE; | (SEQ ID NO. 18) |
| EEE | (SEQ ID NO. 19) |
| or | |
| EE. | (SEQ ID NO. 20) |

Other representative peptides may include any of the peptides of SEQ ID NO. 16 to 20 above wherein one or more of the E residues are replaced by Asp (D) or a Glu derivative, or by any amino acid residue, as long as at least one Glu (E) remains (or other acidic amino acid residue), preferably by an amino acid residue such as Ala (A), Leu (L), Ile (I), Ser (S), Val (V), Gln (Q), Asn (N). Other amino acids which may be substituted include also Gly (G).

Taken together, these various aspects of the invention pertaining to various “active” peptides (i.e. peptides having C-peptide activity) or peptides contributing to C-peptide activity, can be seen to provide a peptide for use in therapy, said peptide being the N-terminal fragment of human proinsulin C-peptide, and having the sequence

| E A E D L Q V G Q V E L | (SEQ ID. NO. 2) |
|---|---|
| 1 2 3 4 5 6 7 8 9 10 11 12 | |

or a fragment or peptide derivative thereof having C-peptide activity or retaining the functional ability of said N-terminal fragment to contribute to C-peptide activity, wherein said fragment or peptide derivative comprises at least one acidic amino acid residue and wherein said peptide derivative does not include native C-peptide of any species (as shown in FIG. 1) nor human C-peptide 1-15, 1-24, 4-24, 7-24,. 11-24, 4-31, 8-31, 12-31 or des 13-17 (C-peptide 1-31 with residues at positions 13-17 missing/deleted). Preferably the peptide is capable of forming an α helix.

This aspect also provides the use of such a peptide in preparing a medicament for use in C-peptide based therapy, pharmaceutical compositions containing such a peptide and methods of treatment (i.e. C-peptide based therapy) wherein such peptides are administered.

The acidic amino acid residues may be the residue of any acidic amino acid, whether naturally occurring or synthetic. Thus, such an amino acid may be any amino acid having a free carboxyl group (or carboxylate etc.). This may be, for example, one of the “conventional” acidic amino acids Glu or Asp or a non-conventional acidic amino acid, such as are well known in the art and widely described in the literature. Such non-conventional acidic amino acids may include modifications or derivatives of Asp or Glu. Acidic D-amino acids may also be used. Representative acidic amino acids are included in the listing of non-conventional amino acids shown in Table 1 below.

TABLE 1

| Non-conventional amino acid | Code |
|---|---|
| α-aminobutyric acid | Abu |
| α-amino-α-methylbutyrate | Mgabu |
| aminocyclopropanecarboxylate | Cpro |
| aminoisobutyric acid | Aib |
| aminonorbornylcarboxylate | Norb |
| cyclohexylalanine | |
| cyclopentylalanine | Cpen |
| D-alanine | Dal |
| D-arginine | Darg |
| D-aspartic acid | Dasp |
| D-cysteine | Dcys |
| D-glutamine | DgIn |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-α-methylalanine | Dmala |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | Dmorn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmty |
| D-α-methylvaline | Dmval |
| D-N-methylalanine | Dnmala |
| D-N-methylarginine | Dnmarg |
| D-N-methylasparagine | Dnmasn |
| D-N-methylaspartate | Dnmasp |
| D-N-methylcysteine | Dnmcys |
| D-N-methylglutamine | Dnmgln |
| D-N-methylglutamate | Dnmglu |
| D-N-methylhistidine | Dnmhis |
| D-N-methylisoleucine | Dnmile |
| D-N-methylleucine | Dnmleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| γ-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-α-methylhistidine | Mhis |
| L-α-methylisoleucine | Mile |
| L-α-methylleucine | Mleu |
| L-α-methylmethionine | Mmet |
| L-α-methylnorvaline | Mnva |
| L-α-methylphenylalanine | Mphe |
| L-α-methylserine | Mser |
| L-α-methyltryptophan | Mtrp |
| L-α-methylvaline | Mval |
| N-(N-(2,2-diphenylethyl) | Nnbhm |
| carbamylmethyl)glycine | |
| 1-carboxy-1(2,2-diphenyl- | Nmbc |
| ethylamino)cyclopropane | |
| L-N-methylalanine | Nmala |
| L-N-methylarginine | Nmarg |
| L-N-methylasparagine | Nmasn |
| L-N-methy1aspartic acid | Nmasp |
| L-N-methylcysteine | Nmcys |
| L-N-methylglutamine | Nmgln |
| L-N-methylglutamic acid | Nmglu |
| Chexa L-N-methylhistidine | Nmhis |
| L-N-methylisolleucine | Nmile |
| L-N-methylleucine | Nmleu |
| L-N-methyllysine | Nmlys |
| L-N-methylmethionine | Nmmet |
| L-N-methylnorleucine | Nmnle |
| L-N-methylnorvaline | Nmnva |
| L-N-methylomithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylethylglycine | Nmetg |
| L-N-methyl-t-butylglycine | Nmtbug |
| L-norleucine | Nle |
| L-norvaline | Nva |
| α-methyl-aminoisobutyrate | Maib |
| α-methyl-y-aminobutyrate | Mgabu |
| α-methylcyclohexylalanine | Mchexa |
| α-methylcylcopentylalanine | Mcpen |
| α-methyl-α-napthylalanine | Manap |
| α-methylpenicillamine | Mpen |
| N-(4-aminobutyl)glycine | Nglu |
| N-(2-aminoethyl)glycine | Naeg |
| N-(3-aminopropyl)glycine | Norn |
| N-amino-α-methylbutyrate | Nmaabu |
| α-napthylalanine | Anap |
| N-benzylglycine | Nphe |
| N-(2-carbamylethyl)glycine | Ngln |
| N-(carbamylmethyl)glycine | Nasn |
| N-(2-carboxyethyl)glycine | Nglu |
| N-(carboxymethyl)glycine | Nasp |
| N-cyclobutylglycine | Ncbut |
| N-cycloheptylglycine | Nchep |
| N-cyclohexylglycine | Nchex |
| N-cyclodecylglycine | Ncdec |
| N-cylcododecylglycine | Ncdod |
| N-cyclooctylglycine | Ncoct |
| N-cyclopropylglycine | Ncpro |
| N-cycloundecylglycine | Ncund |
| N-(2,2-dipheny1ethy1)glycine | Nbhm |
| N-(3,3-diphenylpropyl)glycine | Nbhe |
| N-(3-guanidinopropyl)glycine | Narg |
| N-(1-hydroxyethyl)glycine | Nthr |
| N-(hydroxyethyl))glycine | Nser |
| N-(imidazolylethyl))glycine | Nhis |
| N-(3-indolylyethyl)glycine | Nhtrp |
| N-methyl-γ-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylthreonine | Dmnthr |
| N-(1-methylethyl)glycine | NvalNma |
| N-methyla-napthylalanine | nap |
| N-methylpenicillamine | Nmpen |
| N-(p-hydroxyphenyl)glycine | Nhtyr |
| N-(thiomethyl)glycine | Ncys |
| penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| L-α-methyllysine | Mlys |
| L-α-methylnorleucine | Mnle |
| L-α-methylornithine | Morn |
| L-α-methylproline | Mpro |
| L-α-methylthreonine | Mthr |
| L-α-methyltyrosine | Mtyr |
| L-N-methylhomophenylalanine | Nmhphe |
| N-(N-(3,3-diphenylpropyl) | Nnbhe |
| carbamylmethyl)glycine | |

Preferably the acidic amino acid is Glu or a derivative thereof, e.g. γ-carboxy Glu (Gla), L-N-methyl Glu (Nmglu), L-α-methyl Glu (MGlu).

In a preferred embodiment, the peptide derivatives of the present invention have the formula (I):
Xn-Y-Xₘ-Y-Xₚ (I)
wherein

X is any amino acid;

Y is an acidic amino acid;

n=0-6;

m=1-12; and

p=0-6.

Preferably, the peptide of the invention will have the formula (I), wherein m=5-8 or 5-9.

X may be any amino acid, whether natural or synthetic, conventional or non-conventional. In addition to the well known 20 conventional amino acids (Ala(A); Cys(C); Asp(D); Glu(E); Phe(F); Gly(G); His(H); Ile(I); Lys(K); Leu(L); Met(M); Asn(N); Pro(P); Gln(Q); Arg(R); Ser(S); Thr(T); Val(V); Trp(W); and Tyr(Y)), a number of non-conventional amino acids are shown in Table 1 above.

In particular X may be Ala, Leu or Glu, or any other helix promoting amino acid, or any derivative thereof, e.g. norleucine (Nle).

Further, assuming that the two Y residues in Formula I above correspond to Glu3 and Glu11 of human C-peptide, X may also be any of the amino acid residues which occur in the corresponding positions of human or other (preferably mammalian) C-peptide.

More particularly, in Xₙ, X may be Glu(E), Gln(Q), Asp(D) Asn(N), Ala(A), Val(V), Leu(L), Ser(S), Ile(I) or Gly(G) or a derivative thereof, although preferably at least one X will be a helix promoting amino acid, preferably Ala(A), Leu(L), Nle or Glu(E).

In Xₘ, X may likewise be selected from Glu(E), Gln(Q), Asp(D), Asn(N), Ala(A), Leu(L), Ile(I), Ser(S), Val(V) or Gly(G), but preferably at least one, and preferably at least two, three or four X will be a helix promoting amino acid, preferably Ala(A), Leu(L), Nle or Glu(E).

In Xₚ, X may similarly be selected from Glu(E), Gln(Q), Asp(D), Asn(N), Ala(A), Leu(L), Ile(I), Ser(S), Val(V) or Gly(G), and preferably at least one X will be a helix-promoting amino acid, preferably Ala(A), Leu(L), Nle or Glu(E).

Y may be any acidic amino acid, as defined both generally, and more particularly, above. As mentioned above in preferred embodiments Y is preferably Glu(E) or a derivative thereof.

n is preferably 0-5, 1-5, 1-4, 1-3 or 1-2; e.g. 0, 1, 2, 3, 4, 5 or 6;

m is preferably 1-10, 1-8, 2-8, 3-8, 4-8, 5-8, 5-9 or 6-8; for example 1, 2, 3, 4, 5, 6, 7, 8, 9, 10, 11 or 12;

p is preferably 0-5, 0-4, 0-3, 0-2 or 1-5, 1-4, 1-3 or 1-2, e.g. 0, 1, 2 or 3, 4, 5 or 6.

In more particular embodiments group (Xₙ) may be Y(Xₙ) where Y is any acidic amino acid as defined above, preferably Glu(E), X is as defined above (both particularly and generally) and n is 0-6, preferably 1-3, most preferably 1.

Group (Xₙ) may thus preferably be E-A, E-V-, E-L or E-E, or E-Nle-.

Group (Xₘ) may be
X₍₁₋₄₎-Q-V-X₍₁₋₄₎, e.g. X₍₂₎-Q-V-X₍₃₎;
   X₍₁₋₄₎-Q-X₍₁₋₅₎ e.g. X₍₂₎-Q-X₍₄₎;
X₍₁₋₄₎-Q-A-X₍₁₋₄₎, e.g. X₍₂₎-Q-A-X₍₃₎;
X₍₁₋₄₎-Q-L-X₍₁₋₄₎, e.g. X₍₂₎-Q-L-X₍₃₎;
X₍₁₋₄₎-Q-E-X₍₁₋₄₎, e.g. X₍₂₎-Q-E-X₍₃₎;
X(1-3)-L-Q-X₍₁₋₅₎, e.g. X-L-Q-X₍₄₎;
   wherein X is as defined above, both particularly and generally.

Group (Xₚ) may preferably be L, I, V, A, S, E, Q D, N or G or Nle, preferably A, E, L or Nle, and especially preferably L or Nle.

A representative peptide derivative of the invention may thus be of Formula (II), (III), (IV) or (V):
E A (Y) (X) L Q (X) (X) Q (X) (Y) L (II)
E A (Y) (X) L Q (V) (X) Q (X) (Y) L (III)
(X) (X) (Y) (X) L Q (X) (X) (X) (X) (Y) L (III)
E (X) (Y) (X) L Q (X) (X) (X) (X) (Y) L (IV)
where (X) is any amino acid as defined above (both generally and particularly) and Y is any acidic amino acid, preferably Glu(E) or a derivative thereof, especially Glu(E).

In Formula II, III, IV or V, X is preferably Ala (A), Leu(L), Glu(E) or Nle, or any derivative thereof or other helix promoting amino acid, especially preferably Ala(A), Leu(L), Glu(E) or Nle.

Representative peptides derivatives of the invention thus include;

| | |
|---|---|
| EAEALQAAQAEL | (SEQ ID NO. 21) |
| EAEELQEEQEEL | (SEQ ID NO. 22) |
| EAELLQLLQLEL | (SEQ ID NO. 23) |
| EAE(Nle)LQ(Nle)(Nle)Q(Nle)EL | (SEQ ID NO. 24) |
| AEALQAAQAEL | (SEQ ID NO. 25) |
| EALQAAQAEL | (SEQ ID NO. 26) |
| AEELQEEQEEL | (SEQ ID NO. 27) |
| EELQEEQEEL | (SEQ ID NO. 28) |
| AELLQLLQLEL | (SEQ ID NO. 29) |
| ELLQLLQLEL | (SEQ ID NO. 30) |
| AE(Nle)LQ(Nle)(Nle)Q(Nle)EL | (SEQ ID NO. 31) |
| E(Nle)LQ(Nle)(Nle)Q(Nle)EL | (SEQ ID NO. 32) |
| EAEALQAAQAEA | (SEQ ID NO. 33) |
| EAEALQAAQAE | (SEQ ID NO. 34) |
| AEALQAAQAE | (SEQ ID NO. 35) |
| EALQAAQAE | (SEQ ID NO. 36) |
| EAEALQAAQAEE | (SEQ ID NO. 37) |
| EAEALQAAQAE(Nle) | (SEQ ID NO. 38) |
| AEALQAAQAE(Nle) | (SEQ ID NO. 39) |
| EALQAAQAE(Nle) | (SEQ ID NO. 40) |

Representative derivatives may also include the non-human counterparts of the C-peptide N-terminal segment in other species as shown in FIG. 1, particularly other mammalian species.

The length of the peptide derivatives of the invention is not critical and may, for example, lie in the region of up to 30 residues e.g. up to 26, 24, 20, 16 or 12 residues. Such peptide derivatives may be from e.g. 5, 6, 7 or 8 amino acid residues in length. Representative peptide lengths thus include e.g. 6-26, 6-20, 6-12, 8-12 etc.

Amino acid replacement studies have demonstrated that residues in the N-terminal fragment of C-peptide may be important for activity and that activity may be modulated (e.g. decreased or increased) by substituting certain residues. Thus, activity of human C-peptide may be increased or remain by substituting any of Asp 4, Val 7, Gly 8 and Val 10 with Ala. Preferred peptide derivatives of the invention thus include peptides of SEQ ID NO. 2, wherein one or more of D4, V7, G8 and V10, particularly D4, G8 and V10, are replaced by A.

Further studies have shown that substituting the same residues with other amino acids, in particular helix non-promoters may decrease activity. Thus activity of human C-peptide may be decreased by substituting any of the above residues with Pro. Further preferred peptide derivatives thus include peptides of SEQ ID NO. 2 wherein one or more of D4, V7, G8 and V10 (particularly D4, G8 and V10) are replaced by P or other amino acid residues which do not promote secondary structure e.g. G. Peptides having decreased C-peptide activity may be of value in certain situations. This, thus, opens up the possibility of modulating C-peptide activity by designing peptide derivatives with appropriate substitutions.

As mentioned above, aside from the short active family of peptides and fragments discussed above, the N-terminal fragment and peptide derivatives of the invention as defined above are not active on their own, but contribute to C-peptide activity, e.g. are active in the context of a modified peptide comprising a C-peptide C-terminal portion as required for activity. In other words the N-terminal fragment and peptide derivative sequences may “substitute for” the corresponding native N-terminal segment (sequence) in a native C-peptide molecule. Thus, modified “C-peptides” (or C-peptide sequences or polypeptides) may be constructed which comprise the N-terminal fragment or peptide derivatives of the invention, along with other portions or segments of a C-peptide molecule (e.g. the mid-portion and/or C-terminal segment) and which exhibit C-peptide activity.

Thus, a further aspect of the invention provides a peptide comprising the N-terminal fragment of human proinsulin C-peptide, or a fragment or peptide derivative thereof as defined above, and having C-peptide activity, but not including native C-peptide of any species (as shown in FIG. 1) nor human C-peptide 1-15, 1-24 or des 13-17.

More particularly, this aspect of the invention provides a peptide having an amino acid sequence comprising (i) the N-terminal fragment of human insulin C-peptide having the sequence

| | |
|---|---|
| E A E D L Q V G Q V E L | (SEQ ID NO. 2) |

or (ii) a fragment or peptide derivative of amino acid sequence SEQ ID NO. 2 retaining the functional ability of said N-terminal fragment to contribute to C-peptide activity, wherein said fragment or peptide derivative comprises two acidic amino acid residues and is capable of adopting a conformation wherein said two acidic amino acid residues are spatially separated from one another by a distance of 9-14 Å between the α-carbons thereof, preferably 10-13 Å (e.g. 10-12.6 Å);

said peptide having C-peptide activity, but not including native C-peptide of any species (as shown in FIG. 1) nor human C-peptide 1-15, 1-24 or des 13-17 (C-peptide 1-31 but with residues at positions 13-17 missing).

The spacing between the two acidic amino acids can be 5-9 amino acid residues, preferably 5-8 or 6-8 amino acid residues and most preferably 7 amino acid residues. As indicated above, the peptide is preferably capable of forming an α helix.

C-peptide activity is as defined above.

It will be seen, therefore, that this aspect of the invention provides modified “C-peptides” which have C-peptide activity.

Thus, in this aspect of the invention, an N-terminal C-peptide fragment or derivative may be combined with e.g. the C-terminal pentapeptide of human C-peptide (or its counterpart from other species) to provide a modified C-peptide having C-peptide activity.

Thus, in one such embodiment the N-terminal fragment or derivative of the invention as defined above, may be linked to a C-peptide C-terminal fragment or sequence by a spacer sequence. Such a spacer sequence may comprise all or a portion of a C-peptide mid-portion sequence (the mid-portion being residues 13 to 26 of human C-peptide, or the corresponding segment of another native C-peptide molecule; see FIG. 1) e.g. a truncated mid-portion sequence, a modification or derivative of the mid-portion sequence, e.g. having amino acid substitutions, or a synthetic sequence. Such a synthetic sequence will preferably be composed of amino acids which do not promote secondary structure, e.g. Gly, Pro, Ser, Ile, Val, Asp, Asn and Gln. Gly is preferred. However any amino acid may be used including e.g. Ala or Leu which are helix-promoting. Such a synthetic sequence may be a homopolymeric sequence, e.g. poly Gly. The spacer may also comprise a portion of another segment of the C-peptide molecule. The length of the spacer sequence is not critical (as indeed is its presence).

Accordingly, this may be e.g. up to 15 residues long, preferably 0 to 12, 0 to 10, 0 to 8, 0 to 6, 0 to 5, 0 to 4 or 0 to 3 residues (e.g. 1 to 10, 1 to 18, 1 to 6, 1 to 4, or 1 to 3; or 2 to 8, 2 to 6 or 2 to 4).

Thus, a representative spacer sequence may include various truncations and “deletions” of the human C-peptide mid-portion segment, e.g. human C-peptide 13-17 and 23-26; human C-peptide 13-15 and 23-26; human C-peptide 23-26; human C peptide 23-26 wherein residues 23 and 24 are substituted by Ala. Also included are homopolymers of e.g. Gly having from 1 to 15, e.g. 1 to 12, 1 to 8, 1 to 6 or 1 to 3 residues, or spacers comprising homopolymeric sequences (e.g. poly Gly) coupled to other sequences or residues, e.g. Gly₁₂-AL etc.

The C-terminal segment or sequence may be any C-peptide C-terminal segment or sequence having C-peptide activity e.g. human 27-31 (or its counterpart in other species—see FIG. 1) or a fragment or derivative thereof (see e.g. WO 98/13384).

In another particular embodiment of this aspect, the invention provides a modified mammalian C-peptide which comprises one or more sequence modifications in the N-terminal segment thereof, but wherein amino acid residues Glu3 and Glu11 (or residues corresponding thereto) are conserved.

In such an embodiment, the N-terminal segment may be regarded as residues 1 to 12 of a native C-peptide sequence. In particular, Gln 6 (or a corresponding residue) is also conserved. Leu 5 (or a corresponding residue) may also be conserved. Furthermore, Glu1 and/or Leu 12 (or corresponding residues) may also be conserved.

In particular Asp 4, Val 7, Gly 8 and/or Val 10, or positions corresponding thereto, particularly Asp 4, Gly 8 and/or Val 10 may be modified, for example by replacement with Ala (and/or other amino acids as discussed above e.g. Glu, Leu and Nle). Other residues which may be modified include Glu 1, Ala 2 and/or Gln 9 or their counterparts. Such a modified C-peptide will have C-peptide activity, as defined and discussed above.

Representative examples of such peptides according to these various aspects of the invention thus include:

| | |
|---|---|
| EAEDLQVGQVELGGGGGGGGGGGGGGEGSLQ | (SEQ ID NO. 41) |
| (human 1-12-(G)₁₄-human 27-31) | |
| EAEDLQVGQVELGGGGGGEGSLQ | (SEQ ID NO. 42) |
| (human 1-12-(G)₆-human 27-31) | |
| EAEDLQVGQVELGGGGGGGGGGGGALEGSLQ | (SEQ ID:NO. 43) |
| (human 1-12-(G)₁₂-human 25-31) | |
| EAEDLQVGQVELGGGPGPLALEGSLQ | (SEQ ID NO. 44) |
| (human 1-12, 13-17, 23-31) | |
| EAEDLQVGQVELGGGPLALEGSLQ | (SEQ ID NO. 45) |
| (human 1-12, 13-15, 23-31) | |
| EAEDLQVGQVELLEGSLQ | (SEQ ID NO. 46) |
| (human 1-12, 26-31) | |
| EAEDLQVGQVELEGSLQ | (SEQ ID NO. 47) |
| (human 1-12, 27-31) | |
| EAEDLQVGQVELAAALEGSLQ | (SEQ ID NO. 48) |
| (human 1-12; AA, human 25-31) | |
| EAEDLQVGQVELGGGGGGGGGGGGGGEVARQ | (SEQ ID NO. 49) |
| (human 1-12-(G)₁₄ Rat 27-31). | |

Further representative examples include any of the N-terminal peptide derivatives of SEQ ID NO. 21 to 40 above coupled either directly or via any of the spacer sequences exemplified above (e.g. in SEQ ID NOS. 39 to 46 or 48-49) to human 27-31.

Such modified peptides may also include chimeric C-peptides comprising portions or segments of C-peptide sequence from different species e.g. human 1-12, 13-26, rat 27-31; or human 1-12, rat 13-26, rat 27-31.

Indeed, also may be included chimeric C-peptides wherein different portions or segments of the same C-peptide molecule are combined in non-native order and/or numbers, e.g. human C-peptide (27-31)-(6-12)-(27-31).

The modified peptides of this aspect of the invention preferably do not include the A and/or B chains of proinsulin.

The peptides and fragments and peptide derivatives mentioned above, all subsumed under the general heading of “peptides of the invention” may readily and conveniently be synthesised using known and standard techniques such as are widely known and well described in the literature. Suitable methods include e.g. the well known Merrifield solid phase synthesis method and derivatives thereof.

Also included in the invention are the salts, solvates and esters of the peptides, such as may be prepared and used in accordance with standard pharmaceutical procedures well known in the art.

Thus the peptides of the invention may be presented as pharmaceutically or physiologically acceptable salts e.g. acid addition salts. This may include both organic and inorganic salts such as those prepared for example from acids such as hydrochloric, hydrofluoric, sulfuric, sulfonic, tartaric, fumaric, hydrobromic, glycolic, citric, maleic, phosphoric, succinic, acetic, nitric, benzoic, ascorbic, p-toluenesulfonic, benzene-sulfonic, naphthalenesulfonic, propionic, and the like. Preferably, the acid addition salts are those prepared from hydrochloric acid, acetic acid, or succinic acid. Such salts may be prepared by conventional methods well known to those skilled in the art.

Alternatively the peptide may be converted into a carboxylic acid salt, such as an ammonium or alkali metal salt e.g. a sodium, potassium or lithium salt etc.

As mentioned above, the peptides of the invention have a utility in C-peptide based therapies, that is in the therapy of (i.e. in combatting) any condition which may be alleviated or improved by, or which responds to, C-peptide administration. “Therapy” and “combatting” in this regard include both treatment and prophylaxis. In particular the peptides of the invention (i.e. including the peptide fragments, peptide derivatives and modified peptides defined above etc.) can be used for the therapy of (i.e. for combatting) diabetes and diabetic complications, most notably type 1 diabetes and its complications. As used herein the term “diabetic complications” includes all complications which may be associated with various forms of diabetes, in particular retinopathy, neuropathy and nephropathy. The peptides may thus be used in treatment of type 1 diabetes patients with one or more of the above-mentioned complications, or for preventing or retarding the development of such complications. Thus, the peptides may be used in C-peptide replacement therapy of diabetic patients. As discussed above, the peptides of the invention which are not in themselves active may be used in conjunction with active peptides such as C-peptide itself or a C-terminal fragment of a C-peptide e.g. the C-terminal pentapeptide of human C-peptide. As discussed above, C-peptide has diverse actions which may be mediated through diverse mechanisms. By combining different modified C-peptides or C-peptide fragments in this manner, different effects may be obtained.

A further aspect of this invention thus provides a peptide of the invention as hereinbefore defined, for use in therapy, and in particular in C-peptide based therapy, (e.g. C-peptide replacement therapy in diabetes), and also the use of such a peptide in preparing a medicament for use in C-peptide based therapy (e.g. for combatting diabetes or diabetic complications).

More particularly, this aspect of the invention provides a peptide, being the N-terminal fragment of human insulin C-peptide, and having the sequence

| E A E D L Q V G Q V E L | (SEQ ID. NO. 2) |
|---|---|
| 1 2 3 4 5 6 7 8 9 10 11 12 | |

or a fragment or peptide derivative thereof retaining the functional ability of said N-terminal fragment to contribute to C-peptide activity, wherein said fragment or peptide derivative comprises two acidic amino acid residues and is capable of adopting a conformation where said two acidic amino acid residues are spatially separated from one another by a distance of 9-14 Å between the α-carbons thereof, preferably 10-13 Å (e.g. 10-12.6 Å), wherein said derivative does not include native C-peptide of any species (as shown in FIG. 1) nor human C-peptide 1-15 or 1-24, for use in therapy, and the use of a such peptide in preparing a medicament for use in C-peptide based therapy. The spacing between the two acidic amino acids can be 5-9 amino acid residues, preferably 5-8 or 6-8 amino acid residues and most preferably 7 amino acid residues. Preferably the peptide is capable of forming an α helix.

This aspect of the invention further provides a peptide having an amino acid sequence comprising (i) the N-terminal fragment of human insulin C-peptide having the sequence

| | |
|---|---|
| E A E D L Q V G Q V E L | (SEQ ID NO. 2) |

or (ii) a fragment or peptide derivative of amino acid sequence SEQ ID NO. 2 retaining the functional ability of said N-terminal fragment to contribute to C-peptide activity, wherein said fragment or peptide derivative comprises two acidic amino acid residues and is capable of adopting a conformation where said two acidic amino acid residues are spatially separated from one another by a distance of 9-14 Å between the α-carbons thereof, preferably 10-13 Å (e.g. 10-12.6 Å);

said peptide having C-peptide activity, but not including native C-peptide of any species (as shown in FIG. 1) nor human C-peptide 1-15, 1-24 or des 13-17 for use in therapy, and the use of such a peptide in preparing a medicament for use in C-peptide-based therapy. The spacing between the two acidic amino acids can be 5-9 amino acid residues, preferably 5-8 or 6-8 amino acid residues and most preferably 7 amino acid residues. As indicated above, the peptide is preferably capable of forming an α helix.

Alternatively viewed, this aspect of the invention also provides a method of combatting diabetes or diabetic complications in a patient, said method comprising administering to said patient a peptide of the invention, as defined above.

A further aspect of the invention provides a pharmaceutical composition comprising a peptide of the invention, as defined above, together with at least one pharmaceutically acceptable carrier or excipient.

In this aspect, the peptide of the invention may be a peptide, being the N-terminal fragment of human insulin C-peptide, and having the sequence

| E A E D L Q V G Q V E L | (SEQ ID. NO. 2) |
|---|---|
| 1 2 3 4 5 6 7 8 9 10 11 12 | |

or a fragment or peptide derivative thereof retaining the functional ability of said N-terminal fragment to contribute to C-peptide activity, wherein said fragment or peptide derivative comprises two acidic amino acid residues and is capable of adopting a conformation where said two acidic amino acid residues are spatially separated from one another by a distance of 9-14 Å between the α-carbons thereof, preferably 10-13 Å (e.g. 10-12.6 Å) and wherein said derivative does not include native C-peptide of any species (as shown in FIG. 1) nor human C-peptide 1-15 or 1-24; or it may be a peptide having an amino acid sequence comprising (i) the N-terminal fragment of human insulin C-peptide having the sequence

| | |
|---|---|
| E A E D L Q V G Q V E L | (SEQ ID NO. 2) |

or (ii) a fragment or peptide derivative of amino acid sequence SEQ ID NO. 2 retaining the functional ability of said N-terminal fragment to contribute to C-peptide activity, wherein said fragment or peptide derivative comprises two acidic amino acid residues and is capable of adopting a conformation where said two acidic amino acid residues are spatially separated from one another by a distance of 9-14 Å between the α-carbons thereof, preferably 10-13 Å (e.g. 10-12.6 Å);

said peptide having C-peptide activity, but not including native C-peptide of any species (as shown in FIG. 1) nor human C-peptide 1-15, 1-24 or des 13-17. The spacing between the two acidic amino acids can be 5-9 amino acid residues, preferably 5-8 or 6-8 amino acid residues and most preferably 7 amino acid residues. Preferably, the peptide is capable of forming an α helix.

As mentioned above, in the case of the peptides of the invention which are not themselves active they may be combined with active peptides. Thus such a pharmaceutical composition may comprise in addition a C-peptide or C-peptide fragment having C-peptide activity, such as C-terminal fragments of C-peptide, in particular the human C-terminal pentapeptide. Suitable fragments are further defined in WO98/13384. However, the respective peptides need not both be included in the same composition/medicament and could be administered separately, in separate compositions/medicaments, simultaneously or sequentially. The two fragments may, however, form part of a single molecule.

A further aspect of the invention thus provides a product containing a peptide, being the N-terminal fragment of human insulin C-peptide, and having the sequence

| E A E D L Q V G Q V E L | (SEQ ID. NO. 2) |
|---|---|
| 1 2 3 4 5 6 7 8 9 10 11 12 | |

or a fragment or peptide derivative thereof retaining the functional ability of said N-terminal fragment to contribute to C-peptide activity, wherein said fragment or peptide derivative comprises two acidic amino acid residues and is capable of adopting a conformation where said two acidic amino acid residues are spatially separated from one another by a distance of 9-14 Å between the α-carbons thereof, preferably 10-13 Å (e.g. 10-12.6 Å) and wherein said derivative does not include native C-peptide of any species (as shown in FIG. 1) nor human C-peptide 1-15 or 1-24, together with a peptide having C-peptide activity (particularly a C-terminal C-peptide fragment, e.g. the human C-peptide C-terminal pentapeptide SEQ ID NO. 3) as a combined preparation for simultaneous, separate or sequential use in C-peptide based therapy (e.g. in combatting diabetes and/or diabetic complications). The spacing between the two acidic amino acids can be 5-9 amino acid residues, preferably 5-8 or 6-8 amino acid residues and most preferably 7 amino acid residues. Preferably, the peptide is capable of forming an α helix.

The peptides may also be used in combination or conjunction with other agents active or effective to treat diabetes and/or its complications. Such other active agents include for example insulin. In such “combination” therapies the peptide(s) and second active agent may be administered together in the same composition or separately in separate compositions, simultaneously or sequentially.

A further aspect of the invention thus provides a product containing a peptide of the invention as hereinbefore defined, together with a further active agent effective to combat diabetes or diabetic complications, as a combined preparation for simultaneous, separate or sequential use in combatting diabetes and/or diabetic complications.

Preferably such a further active agent is insulin.

In such combined therapies, where insulin is used, it is to be understood that the term “insulin” encompasses all forms, types and derivatives of insulin which may be used for therapy e.g. synthetic, modified, or truncated variants of the active human insulin sequence.

The compositions of the invention may be administered in any convenient way, e.g. orally or parenterally, for example by the subcutaneous, intramuscular or intravenous route. The compositions of this invention may comprise active peptides of the invention, together with a pharmaceutically acceptable carrier therefor and optionally, other therapeutic ingredients, for example human insulin. The total amount of active ingredients in the composition may vary from 99.99 to 0.01 percent of weight. The carrier must be acceptable in the sense that it is compatible with other components of the composition and is not deleterious to the recipient thereof.

The compositions may be formulated according to techniques and procedures well known in the art and widely described in the literature, and may comprise any of the known carriers, diluents or excipients. Thus, for example, compositions of this invention suitable for parenteral administration conveniently comprise sterile aqueous solutions and/or suspensions of the pharmaceutically active ingredients (e.g. the peptides of the invention) preferably made isotonic with the blood of the recipient, generally using sodium chloride, glycerin, glucose, mannitol, sorbitol, and the like. In addition, the compositions may contain any of a number of adjuvants, such as buffers, preservatives, dispersing agents, agents that promote rapid onset of action or prolonged duration of action and the like.

Compositions of this invention suitable for oral administration may, for example, comprise the peptides in sterile purified stock powder form preferably covered by an envelope or envelopes (enterocapsule) protecting from degradation (dicarboxylation or hydrolysis) of the peptides in the stomach and thereby enabling absorption of these substances from the gingiva or in the small intestine. The envelope(s) may contain any of a number of adjuvants such as buffers, preservative agents, agents that promote prolonged or rapid release giving an optimal bioavailability of the compositions in this invention, and the like.

Appropriate representative formulations (i.e. compositions) and dosages etc. are described in WO. 98/13384.

The invention will now be described in more detail in the following non-limiting Examples, and with reference to the drawings in which:

FIG. 1 is an alignment showing all reported C-peptide amino acid sequences.

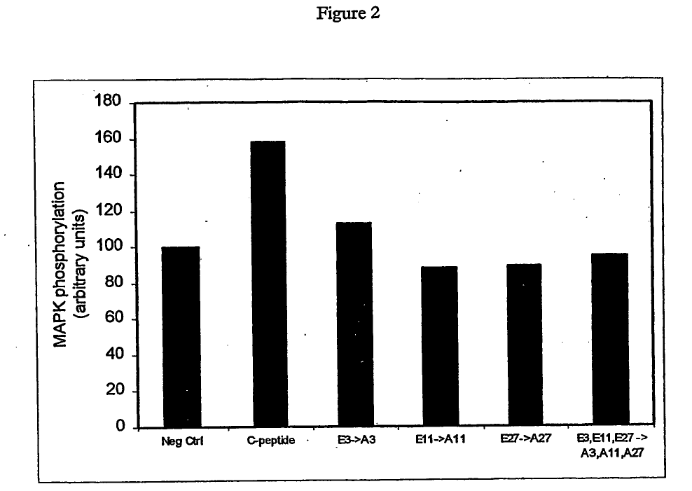

FIG. 2 shows the ability of the various C-peptide mutants from Example 4 to phosphorylate MAPK.

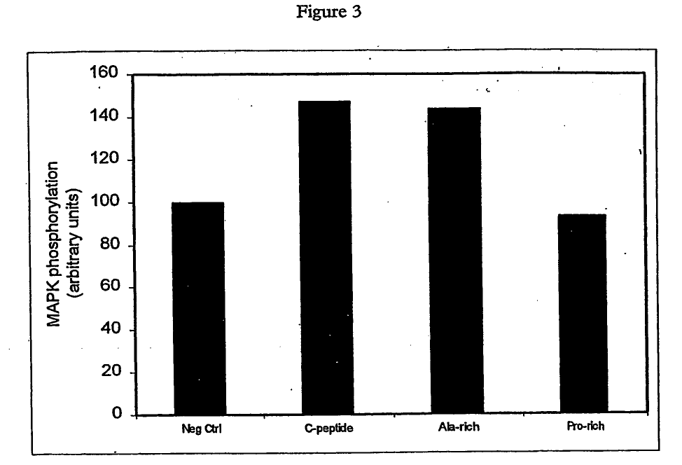

FIG. 3 shows the ability of C-peptide to phosphorylate MAPK when residues 4, 7, 8 and 10 are all altered to either Ala or Pro, where Ala may keep the tendency of the native C-peptide tendency to induce α-helix formation and induce α-helix formation and Pro may prevent α-helix formation.

### EXAMPLES

### Example 1

### Intracellular Ca²⁺ Concentration ([Ca²+]i) Measurements as an Assay of C-peptide Activity

Based on studies showing a C-peptide effect on the intracellular Ca²⁺ concentration ([Ca²⁺]) (Ohtomo et al., supra, Kunt et al., supra), an assay has been developed, for C-peptide activity, based on measuring [Ca²⁺]ᵢ changes in human renal tubular cells, namely a rise in [Ca²⁺]ᵢ.

Peptides

The following peptides were used, all prepared by solid-phase peptide synthesis and purchased from Sigma (Genosys (Cambridge, UK): human C-peptide, scrambled C-peptide (with the same residues, but randomly ordered des (27-31) C-peptide and C-terminal pentapeptide (EGSLQ).

Cell Culture

Human renal tubular cells were obtained from the outer cortex of renal tissues obtained from non-diabetic patients undergoing elective nephrectomy for renal cell carcinomas. The collection of the tissue samples during suegery was approved by the Ethics Committee of the Karolinska Hospital. The cells were cultured in RPMI 1640 (Life Technologies, Grand Island, N.Y.) supplemented with 10% fetal calf serum, 2 mM L-glutamine, 10 mM HEPES, benzylpenicillin (100 U/ml), and streptbmycin (100 U/ml). Cells were grown to confluency on cover slips in a six-well plate and starved in serum-free medium for 4 hours before experiments.

Measurements of [Ca²⁺]ᵢ

Changes in [Ca²⁺¹]₁ were measured on cells attached to cover slips. The cells were loaded with 2 μM of the fluorescent Ca²⁺ indicator fura-2/AM at 37° C. for 30 minutes in a buffer containing 115 mM NaCl, 24 mM NaHCO₃, 4.7 mM KCl, 1.26 mM CaCl₂ 1.2 mM KH₂PO₄, 1.2 mM MgSO_{Z} 11.1 mM glucose, 20 mM HEPES, and 5 mg/ml bovine serum albumin, pH 7.4. Cover slips were mounted as the bottom of an open chamber placed on a thermostatically controlled stage of an inverted epifluorescence microscope (Zeiss, Axiovert 135). The microscope was connected to a SPEX fluorolog-2 system for dual-wave-length excitation fluorimetry. The emissions of the two excitation wavelengths of 340 and 380 nm were used to calculate the fluorescence ratio (F340/F380) reflecting changes in [Ca²⁺]ᵢ, [17]. Treatments during recordings were made using a perifusion system attached to the chamber. Changes in [Ca²⁺]ᵢ were measured using an attached CCD camera that allowed recording of individual cells in the cell clusters. The influence of pertussis toxin was studied by preincubation of the cells with the toxin at 1 μg/ml for 4 h at 37° C.

Results

Human renal tubular cells loaded with the indicator fura-21AM were exposed in different series of experiments to the peptides studied. Changes in [Ca²⁺]ᵢ were recorded. The effect of 5 nM human C-peptide may be seen both as a F340/F380 ratio trace and as images of the cells taken at different time points. The most reproducible responses were obtained at 5 and 10 nM. Hence, comparative measurements were performed at these peptide concentrations. Results similar to those with human C-peptide were obtained with the C-terminal pentapeptide of the human C-peptide, while scrambled C-peptide and des(27-31) C-peptide, lacking the C-terminal pentapeptide part of C-peptide, gave no effect. Successive exposures of the same cells to 5 nM of human C-peptide, a scrambled C-peptide, and the C-terminal pentapeptide, EGSLQ, establish the specificity of the C-peptide effect.

Preincubation of the cells with pertussis toxin at 1 μg/ml eliminated the responses with both C-peptide and the C-terminal pentapeptide. This confirms that the Ca²⁺ effect is dependent on G-protein-coupled signal transduction pathways. When cells were perfused with a buffer without Ca²⁺, the effects of the peptides were abolished, indicating that extracellular Ca²⁺ is required for the response.

### Example 2

### Western Blot Analysis of MAPK Activation as an Assay of C-peptide Activity

Cell Culture and Treatments:

We have used HRTC (human renal tubular cells) isolated in our laboratory from outer cortex of renal tissue obtained from patients undergoing elective nephrectomy. Cells were cultivated in RPMI medium supplemented with 10% FBS, glutamine, Hepes and penicillin streptomycin. We have always used P3 (passage 3). After cells were grown to confluence, they were cultured in serum-free medium for 2-24 h to render them to be quiescent. The cells were then treated with either C-peptide, or controls with vehicle at 37° C. Subsequently, the cells were washed twice with PBS and lysed in ice-cold lysis buffer (50 mM Hepes the lysate was kept on ice foe 30 mm and centrifugated at 12000×g for 10 mm. The supernatant was stored at −80° C. Protein concentration were determined by the method of Lowry using bovine serum albumin as standard.

Western Blot Analysis:

Was performed using 2.5-25 ug protein per lane in 10% SDS gel depending on which protein we want to detect.

### Example 3

### Experiments Showing Functional Importance of Glu 3, Glu 11 and Glu 27 in Human C-peptide

The peptides used, as shown in Table 2, below were prepared by solid phase peptide synthesis. In these peptides each or all of Glu 3, Glu 11 and Glu 27 of human C-peptide were replaced by Ala. In addition, all of Glu 3, Glu 11 and Glu 27 were replaced by Gln. The peptides were tested for C-peptide activity by monitoring for rises in [Ca²⁺]ᵢ as described in Example 1. The following results were obtained:

TABLE 2

| | C-peptide activity in |
|---|---|
| Peptide | [Ca²⁺]ᵢ assay |
| Glu 3 Ala | − |
| Glu 11 Ala | +/− |
| Glu 27 Ala | +/− |
| Glu 3, 11, 27 Ala | − |
| Glu 3, 11, 27 Gln | − |

The results confirm the essentiality of Glu 27 as reported in Pramanik et al., supra and establish the essentiality of Glu 3 and Glu 11.

### Example 4

### Importance of Glutamic Acid Residues Within the C-peptide Primary Structure for its Biological Activity

Swiss 3T3 cells, a rat fibroblast cell line, passage 6, were seeded 96 h before use in three 6-well plates. After 96 h, the cells in each well were confluent. The cultivation medium was Dulbecco's Modified Eagle Medium (DMEM) without sodium pyruvate, with pyridoxineHCl, 4500 mg/l glucose, 50 μg/ml gentamicin, and 10% foetal calf serum (FCS).

The cells were washed thoroughly with 37° C. starvation medium (cultivation medium as above without addition of FCS) and then kept in 1 ml 37° C. starvation medium for 2.5 h. 15 min. prior to the experiment, the buffer was changed to 37° C. Hanks' buffer (Gibco), and then kept in 37° C.

Human C-peptide and the four mutants (i) E₃→A₃; (ii) E₁₁→A₁₁ (iii) E₂₇→A₂₇ and (iv) E₃→A₃, E₁₁→A₁₁, E₂₇→A₂₇ were diluted to 1 nM in Hanks' buffer.

The cells in the wells were stimulated for 3 minutes at 37° C. with 1 ml each of the 1 nM peptide solutions. As a negative control, certain wells were treated with Hanks' buffer only. Each peptide solution and the negative control were applied in three separate wells.

Following stimulation of the cells with the peptide solutions, the cells were washed 3 times with ice-cold Hanks' buffer. All buffer in the well was removed, and the cells were lysed with 200 μl ice-cold lysis buffer (50 mM HEPES (pH 7.5), 150 mM NaCl, 5 mM EDTA, 10 mM sodium pyrophosphate, 2 mM NaVO₃, 1×Complete™ protease inhibitor, and 1% (v/v) Nonidet P40).

The cell lysates were kept in the dishes on ice for 30 min. The lysate was removed with a rubber policeman, transferred to Eppendoff tubes, homogenised (vortex), and kept on ice for 60 min. The tubes were vortexed and centrifuged for 15 min at 16 000×g in the cold room. The supernatants were transferred into new Eppendorf tubes and kept on ice.

Protein content was determined in the collected supernatants using a BCA Protein Assay Reagent Kit (Pierce), including albumin standards. The collected samples had a protein concentration in the range of 806-1071 μg/ml. Samples for SDS-PAGE were prepared by standardizing the protein content to 600 μg/ml in a SDS-PAGE sample buffer. Prepared samples (10 μl, 6 μg protein) were subjected to SDS-PAGE in 10% Tris-glycine gels (Novex) and transferred to 0.2 μm PVDF membranes (Novex) using standard procedures. The membranes were blocked with 5% fat-free milk powder in TBS/T (Tris-buffered saline+0.1% Tween-20) for 1 h at room temperature.

The PVDF membranes were incubated with primary antibodies against (I) Phosphop44/42 MAP Kinase (Thr202/Tyr204) and (II) p44/42 MAP Kinase, respectively. The Phospho-p44/42 MAP Kinase (Thr202/Tyr204) antibody detects p42 and p44 MAP kinase (Erk1 and Erk2) only when catalytically activated by phosphorylation at Thr202 and Tyr204 of human Erk, or Thr183 and Tyr185 of rat Erk. Both antibodies are commercially available, polyclonal antibodies from rabbits (Cell Signalling Technology, #9101 and #9102, respectively). Following incubation with the primary antibodies, diluted 1:1000, in TBS/T overnight at 4° C. on a rocking table, the PVDF membranes were rinsed and incubated with a secondary antibody, (goat anti-rabbit IgG (H+L) HRP (horseradish peroxidase) conjugate, diluted 1:20000) in TBS/T for 1 h at room temperature on a rocking table. The membranes were rinsed with TBS/T, and HRP activity on the membranes was detected with the SuperSignal® West Pico Chemiluminescent Substrate kit (Pierce).

Photographic films were exposed to the membranes, and bands of appropriate sizes on the resulting films were quantitated by densitometry, using a GS-710 Imaging Densitometer (Bio-Rad) and Quantity One software (Bio-Rad). The averaged results are displayed in FIG. 2.

Notably, substitution of any one of the three glutamic acid residues at positions 3, 11, and 27 results in a decreased phosphorylation of p44/42 MAP kinase by the corresponding C-peptide mutant. The decrease in phosphorylation is indicative of a decreased biological activity. Possibly, the glutamic acid residues at positions 11 and 27 are more important than the glutamic acid residue at position 3. Consequently, the mutant carrying three mutations (E₃→A₃, E₁₁→A11, E₂₇→A₂₇) also displays a loss of phosphorylating activity in this assay.

### Example 5

### Importance of N-terminally Located Amino Acid Residues for Potential C-peptide Secondary Structure

The following peptides were analysed by circular dichroism (CD) spectroscopy (data not shown) in 10 mM phosphate buffer, pH 7, and in 50% (vol/vol) trifluoroethanol in phosphate buffer, pH 7:

| | |
|---|---|
| C-peptide | EAEDLQVGQV ELGGGPGAGS LQPLALEGSL Q |
| Alanine-rich | EAEALQAAQA ELGGGPGAGS LQPLALEGSL Q |
| C-peptide | |
| Proline-rich | EAEPLQPPQP ELGGGPGAGS LQPLALEGSL Q |
| C-peptide | |

The resulting CD spectra reveal that all three peptides lack secondary structure in phosphate buffer. In the presence of 50% trifluoroethanol, C-peptide has a tendency to form a partially α-helical structure. This tendency is even more pronounced in the alanine-rich C-peptide, while the lack of secondary structure remains in the proline-rich C-peptide.

Trifluoroethanol disfavors intramolecular hydrogen binding between peptide and solvent, and can be employed to study the tendency of a peptide to adopt a stable secondary structure, e.g. as a part of a protein. These results also imply that the induced α-helix is formed in the first ten amino acid residues, since proline substitutions can prevent α-helix formation, and alanine substitutions can further induce α-helix formation.

### Example 6

### Importance of N-terminally Located Amino Acid Residues for C-peptide Biological Activity

Swiss 3T3 cells, passage 6, were seeded 96 h before use in three 6-well plates. After 96 h, the cells in each well were nearly confluent. The cultivation medium was Dulbecco's Modified Eagle Medium (DMEM) without sodium pyruvate, with pyridoxineHCl, 4500 mg/l glucose, 50 μg/ml gentamicin and 10% foetal calf serum (FCS).

The cells were washed thoroughly with 37° C. starvation medium (cultivation medium as above without addition of FCS) and then kept in 1 ml 37° C. starvation medium for 2.5 h. Prior to the experiment, the buffer was changed to 37° C. Hanks' buffer (Gibco), and then kept in 37° C.

The cells in the wells were stimulated for 3 minutes at 37° C. with 2 ml each of the peptides of Example 5, peptide concentration 1 nM. As a negative control, certain wells were treated with Hanks' buffer only. Each peptide solution and the negative control were applied in three separate wells.

Following stimulation of the cells with the peptide solutions, the cells were washed with ice-cold Hanks' buffer. All buffer in the well was removed, and the cells were lysed with 200 μl ice-cold lysis buffer (50 mM HEPES (pH 7.5), 150 mM NaCl, 5 mM EDTA, 10 mM sodium pyrophosphate, 2 mM NaVO₃, 1×Complete™ protease inhibitor, and 1% (v/v) Nonidet P40).

The cell lysates were kept in the dishes on ice for 80 min. The lysate was removed with a rubber policeman, transferred to Eppendoff tubes, homogenised (vortex), and kept on ice for 30 min. The tubes were vortexed and centrifuged for 15 minutes at 16 000×g the cold room. The supernatants were transferred into new Eppendorf tubes, kept on ice.

Protein content was determined in the collected supernatants using a BCA Protein Assay Reagent Kit (Pierce), including albumin standards. The collected samples had a protein concentration in the range of 3 28-667 μg/ml. Samples for SDS-PAGE were prepared by standardizing the protein content to 248 μg/ml in a SDS-PAGE sample buffer. Prepared samples (approximately 5 μg protein) were subjected to SDS-PAGE, transferred to PVDF membranes, and the membranes were subjected to primary and secondary antibodies, as outlined in Example 1. HRP activity on the membranes was detected by chemoluminescense, and photographic films were exposed to the membranes. Bands of appropriate sizes on the resulting films were quantitated by densitometry, using a GS-710 Imaging Densitometer (BioRad) and Quantity One software (Bio-Rad). The results are displayed in FIG. 3.

In this assay, substitution of native residues for alanine residues in positions 4, 7, 8, and 10 did not decrease the biological activity of C-peptide, measured as capacity of phosphorylating p44/42 MAP kinase. However, substitution of native residues for proline residues in positions 4, 7, 8, and 10 resulted in a decrease in phosphorylating capacity of C-peptide. Therefore the capacity for form an N-terminally situated helix is important for full C-peptide activity.

1. A peptide, being the N-terminal fragment of human proinsulin C-peptide, and having the sequence

| E A E D L Q V G Q V E L | (SEQ ID. NO. 2) |
|---|---|
| 1 2 3 4 5 6 7 8 9 10 11 12 | |

or a fragment or peptide derivative thereof retaining the functional ability of said N-terminal fragment to contribute to C-peptide activity, wherein said fragment or peptide derivative comprises two acidic amino acid residues and is capable of adopting a conformation where said two acidic amino acid residues are spatially separated from one another by a distance of 9-14 Å between the a-carbons thereof; and wherein said peptide derivative does not include native C-peptide of any species nor human C-peptide 1-15, 1-24 or 1-26 or rat Cpeptide 1-26.

2. A peptide having an amino acid sequence comprising (i) the N-terminal fragment of human insulin C-peptide having the sequence

| | |
|---|---|
| E A E D L Q V G Q V E L | (SEQ ID NO. 2) |

or (ii) a fragment or peptide derivative of amino acid sequence SEQ ID NO. 2 retaining the functional ability of said N-terminal fragment to contribute to C peptide activity, wherein said fragment or peptide derivative comprises two acidic amino acid residues and is capable of adopting a conformation wherein said two acidic amino acid residues are spatially separated from one another by a distance of 9-14 Å between the acarbons thereof;
said peptide having C-peptide activity, but not including native C-peptide of any species nor human C-peptide 1-15, 1-24 or des 13-17.

3. A peptide according to claim 1 having the formula (I):
Xn-Y-Xm-Y-XP (I)
wherein
X is any amino acid;
Y is an acidic amino acid;
n=0-6;
m=5-9; and
p=0-6.

4. The peptide of claim 3, wherein m is 5-8.

5. The peptide of claim 3, wherein m is 7.

6. The peptide of claim 1 which is capable of adopting an α-helical conformation.

7. The peptide of claim 6 wherein said two acidic amino acid residues are located on one side of said α-helix.

8. The peptide according to claim 6 which is a peptide derivative of SEQ. ID. No. 2 but comprises further amino acid residues of the N-terminal fragment of C-peptide (SEQ. ID. No. 2) which are located on one side of said a-helix such that the helix presents a conserved surface.

9. The peptide of claim 8 wherein said conserved surface comprises Gln 6 and/or Val 7 in addition to said two acidic residues.

10. The peptide of claim 1 further comprising a third acidic amino acid residue capable of interacting with said two acidic amino acid residues.

11. The peptide of claim 1 wherein at least one of the acidic amino acid residues is Glu.

12. The peptide of claim 11 wherein said two acidic amino acid residues are Glu.

13. A salt, solvate or ester of the peptides of claim 1.

14. The peptide of claim 1 wherein said two acidic amino acid residues are spatially separated from one another by a distance of 10-13 Å between the α-carbons thereof.

15. A peptide, being the N-terminal fragment of human insulin C-peptide and having the sequence
E A E D L Q V G Q V E L (SEQ ID NO. 2)
or a fragment or peptide derivative thereof retaining the functional ability of said N-terminal fragment to contribute to C-peptide activity, wherein said fragment or peptide derivative comprises two acidic amino acid residues and is capable of adopting a conformation where said two acidic amino acid residues are spatially separated from one another by a distance of 9-14 Å between the α-carbons thereof, wherein said derivative does not include native C-peptide of any species nor human C-peptide 1-15 or 1-24, for use in therapy.

16. A peptide having an amino acid sequence comprising (i) the N-terminal fragment of human insulin C-peptide having the sequence
E A E D L Q V G Q V E L (SEQ ID NO. 2)
or (ii) a fragment or peptide derivative of amino acid sequence SEQ ID No. 2 retaining the functional ability of said N-terminal fragment to contribute to C peptide activity, wherein said fragment or peptide derivative comprises two acidic amino acid residues and is capable of adopting a conformation where said two acidic amino acid residues are spatially separated from one another by a distance of 9-14 Å between the 60 -carbons thereof;
said peptide having C-peptide activity, but not including native C-peptide of any species nor human Cpeptide 1-15, 1-24 or des 13-17 for use in therapy

17. The peptide of claim 15, wherein said therapy is C-peptide based therapy.

18. The peptide of claim 15, used in conjunction with C-peptide or a C-terminal fragment of C-peptide.

19. The peptide claim 15, wherein said peptide further comprises a C-terminal fragment of C-peptide.

20. The peptide of claim 18, wherein the Cterminal fragment of C-peptide is EGSLQ.

21. Use of a peptide as defined in claim 15 in the manufacture of a medicament for use in C-peptide based therapy.

22. A pharmaceutical composition comprising a peptide as defined in claim 15 together with a pharmaceutically acceptable carrier or excipient.

23. The pharmaceutical composition of claim 22 further comprising a C-peptide or C-peptide fragment having C-peptide activity.

24. A product containing the peptide of claim 1, together with a peptide having C-peptide activity, as a combined preparation for simultaneous, separate or sequential use in C-peptide based therapy.

25. A product as claimed in claim 24 wherein said peptide having C-peptide activity is a C-terminal fragment of human C-peptide.
